(19) United States
(12) Patent Application Publication
(10) Pub. No.: US 2005/0038345 A1
(43) Pub. Date: Feb. 17, 2005

(54) Apparatus and method for non-invasive monitoring of heart performance

(76) Inventors: Nora Viviana Gorgenberg, Haifa (IL); Hector Rotstein, Haifa (IL); Michael Narodnitsky, Carmiel (IL); Alon Marmor, Zfat (IL); Miguel Gorenberg, Haifa (IL)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/070,940

(22) PCT Filed: Jun. 27, 2001
(86) PCT No.: PCT/IL01/00583

(30) Foreign Application Priority Data
Jun. 27, 2000 (IL) 137045

(51) Int. Cl. A61B 5/02

(52) U.S. Cl. 600/485; 600/502; 600/504; 600/526

(57) ABSTRACT

A non-invasive and portable apparatus is provided in order to monitor parameters indicative of heart performance, such as blood flow, and comprises at least one sensor adapted to continuously sense factors correlated with blood flow and collect data related to the flow of blood, the sensor is adapted to be positioned adjacent to a peripheral blood vessel and worn preferably on a wrist. The apparatus further comprises a processor adapted to receive the collected data from the sensor and calculate the parameters indicating heart performance and a monitor on which the parameters indicating heart performance are displayed.

### FIELD OF THE INVENTION

The present invention is related to non-invasive monitoring of heart mechanical performance. More particularly, the present invention is related to a noninvasive apparatus and method for measuring the mechanical performance of the heart using beat-to-beat continuous monitoring and recording the flow of blood using peripheral mounted arterial sensors.

### BACKGROUND OF THE INVENTION

Heart muscle ischemia due to coronary artery diseases is one of the leading causes of death in the world; in the United States only, it affects more than 13 million people. Myocardial ischemia can be defined as a decrease in the supply of blood to the heart, and more precisely as an imbalance between the supply and demand of myocardial oxygen. In most clinical situations, the reason for this imbalance is inadequate perfusion of the myocardium due to obstructions or stenosis of the coronary arteries. The ischemia can last a few seconds or persist for minutes or even hours, causing transient or permanent damage to the heart muscle. The population that suffers ischemic heart diseases is at high risk of recurrent myocardial infraction. Each year, an estimated amount of 2 million Americans will have a new or recurrent coronary attack while more than 40% of the people experiencing coronary attack are expected to die from it.

In order to monitor ischemic incidents and especially recurring ones, population at risk may connect to a cardiac center through a telephone line. Today, ambulatory monitoring of these patients or elderly population is performed using transtelephonic electrocardiography (TTE). Patients experiencing suspected symptoms can correlate these symptoms with their ECG's at the time they are experiencing the incident and than transmit their ECG through the telephone line to the cardiac center.

There are several disadvantages in using TTE:
1. TTE requires the patient to be symptomatic when experiencing a cardiac event. However, 40-70% of transient ischemic episodes are silent, not associated with anginal chest pain or any other symptoms. A patient experiencing a silent episode will most probably not be aware of his situation and consequently will not use the TTE.
2. TTE requires the patient to connect electrodes to his body, activate a recorder, and at the same time to phone the cardiac center and transtelephonic transmit the ECG. This is a complicated and an error-prone procedure, especially when performed by symptoms suffering patient.
3. the ECG test was shown in studies to have low sensitivity for diagnosis of ischemia (about 60%). It has been shown that even patients with clear symptomatology may have a normal ECG.

Experimental and clinical studies in the cardiologic literature and other references indicate that changes in the cardiac mechanical performance occur relatively early when an incidence of ischemia takes place, and indexes reflecting the mechanical performance of the heart are more sensitive than the ECG; hence, subjective symptoms for detecting myocardial ischemia. The following references are disclosed as references: Kayden et al., “Validation of Continuous Radionucleide Left Ventricular Functioning Monitoring in Detecting Silent Myocardial Ischemia during Balloon Angioplasty of the Left Anterior Descending Coronary Artery”, Am. J. Cardiol. 67, 1339-1343 (1991); Kayden et al., “Silent Left Ventricular Dysfunction during Routine Activity after Thrombolitic Therapy for Acute Myocardial Infarction”, J. Am. Coll. Cardiol. 15, 1500-1507 (1990); Tamaki et al., “Continuous Monitoring of Left ventricular function by an Ambulatory Radionucleide Detector in Patients with Coronary Artery Disease”, J. Am. Coll. Cardiol. 12, 669-679 (1988); Breisblatt et al., “Usefulness of Ambulatory Radionucleide Monitoring of Left Ventricular Function Early after Acute Myocardial Infarction for Predicting Residual Myocardial Ischemia”, Am. J. Cardiol. 62, 1005-1010 (1988); Mohiuddin et al., “clinical Evaluation of Cardiac Function by Ambulatory Scintigraphic Monitoring (VEST), Am. Heart J. 123, 386 (1992); Grover et al., “Dissociation between Regional Myocardial Dysfunction and Subendocardial ST Segment Elevation During and After Exercise-Induced Ischemia in Dogs”, J. Am. Coll. Cardiol. 10, 105-112 (1987).

It had become needed, especially among the population at risk, a device that could monitor cardiac mechanical performance continuously and independently of clinical symptomatology. There is limited information regarding the mechanical performance of the heart in acute ischemia that is partially shown in the above-mentioned studies and was obtained using invasive, time limited cardiac catheterization or non-invasively by short-term single dose injection radioisotopes studies or by echocardiography procedures performed in cardiac units in hospitals.

Rough noninvasive measurements of blood flow are performed using Doppler technique in which ultrasonic sound waves are transmitted through the skin roughly parallel to the blood flow direction. Changes in sound transmit time due to the flow of blood is used to determine the blood flow velocity. The Doppler technique has several inherent limitations: the measurement measures blood flow velocity in velocity units rather than the desired volumetric blood flow quantity which is in volume per time units. In addition, signal-to-noise considerations limit the accuracy of the measurement. The measurement is dependent on the location of the sensor in respect with the blood vessel and in order to establish an accurate measurement, the patient should not move during the measurement. As opposed to Doppler sensors, electromagnetic blood meters have the advantage of insensitivety to movements and to contact angle. Moreover, the sensor in the electromagnetic blood meter is suitable for miniaturization, so that the meter is compact and comfortable. A sophisticated device that tends to improve signal-to-noise ratios is disclosed in U.S. Pat. No. 4,412,545 by Okino et al. “Electromagnetic Blood Flowmeter”. In this electromagnetic blood flowmeter, the blood flow is excited by an alternating rectangular magnetic field with a non-excitation period.

While the technique of using a blood flow sense probe around the blood vessel is more preferable than cutting the blood vessel and installing a flow sensor in the blood flow path, it still involves cutting though the skin so that the sensor can be mounted about the blood vessel.

A proper noninvasive measurement is a transcutaneous measurement in which disruption of the vessels or the skin is avoided. This can be achieved by generating an external varying magnetic field, directing it through the skin and the blood vessel and measuring the part of the electric field resulting from blood flow that is available at the skin. The current available noninvasive electromagnetic blood flow sensors have poor signal-to-noise ratios because only a small portion of the magnetic field energy generated externally to the skin is coupled through the blood vessel and only a small portion of the electric magnetic filed generated by the blood flow appears at the skin surface. The result is limited accuracy and excessive sensitivity to noise and motion artifacts.

A noninvasive blood flow measurement is described by Kanai et al., “Transcutaneous Blood Flow Measurement by Electromagnetic Induction”, IEEE Transactions on Biomedical Engineering, BME-21 (2), 1974. In this report, a magnetic unipole that is excited at 400 Hz was placed adjacent to a blood vessel whose blood flow is measured. The difference in the voltage between the electrodes on the skin surface at opposite sides of the blood vessel provides signal amplitude proportional to the blood flow. Also here, the transcutaneous measurement faced difficulties arising from electromagnetic induction, especially electrostatic, leakage resistance and electromagnetic coupling between the detecting and the excitation circuits. An improved sensor is disclosed in U.S. Pat. No. 5,935,077 “Noninvasive Blood Flow Sensor Using Magnetic Field Parallel to Skin” by Ogle and filed in 1997. The improved magnetic blood flow sensor uses a bipolar magnetic field source to provide a varying magnetic field with a component parallel to the skin and through the blood vessel, a single sense electrode on the skin adjacent to the blood vessel, a reference electrode, and a detector that samples the sense electrode signal in synchronism to the varying magnetic field. Still, this sensor is sensitive to its placement and motion so that its accuracy is damaged. There is a need for a noninvasive sensor that is insensitive to the relative skin to sensor displacement and motion and is compact enough so that comfortable measurement conditioned may be met and the mechanical performance of the heart may be monitored.

It is preferable and comfortable to place such monitor on a patient's limb. It has been shown by the inventors that there is an excellent correlation between blood flow and cardiac output that are measured centrally (at the aortic valve) and reflect the cardiac mechanical performance, and the peripheral blood flow and peripherial cardiac output that are measured at the brachial artery. The inventors measured the central ascending aortic flow simultaneously with the brachial flow in patients using two Doppler transducers positioned at the chest (apical fourth chamber view) and at the brachial artery. The measurements were performed at a baseline and after a pharmacological effort with Dobutamine infusion. In the comparison of the change in flow from rest to maximal pharmacological effort between the chest measurement and the brachial measurement, a correlation factor of 0.94 was found, which indicate an excellent correlation between central and peripheral measurement.

Along peripheral blood flow, other hemodynamic indexed of cardiac performance such as peripheral stroke volume (PSV), peripheral cardiac output (CO) and peripheral stroke work (PSW) may provide an indication on the continuous condition of a patient, especially for population that is in risk such as patients that suffer from ischemia or coronary artery diseases.

### SUMMARY OF THE INVENTION

It is an object of the present invention to provide a new and unique noninvasive device and method for monitoring continuously the heart mechanical performance. Among the indexes that reflects the cardiac performance, one may find peripheral stroke volume (PSV), peripheral cardiac output (CO) and peripheral stroke work (PSW)

It is another object of the present invention to provide a new and unique device and method for monitoring the mechanical performance of the heart while the device is preferably wrist-mounted so that comfortable measurements conditions are met. The device may be mounted on another peripheral organ or area that meets the requirements of which blood flow may be measured without interference.

It is an additional object of the present invention to provide a new device that alerts patents to seek for immediate medical assistance when their heart performance is deteriorating.

It is yet another object of the present invention to provide a new device that facilitates true diagnosis in cases of ischemia so that false positive ECG interpretation is avoided.

An additional object of the present invention is to provide a new device and method that facilitates evaluation of ischemia severity.

Yet, it is an additional object of the present invention to provide a new and unique device and method for recording and storing synchronized ECG signals with parameters that are correlated to the mechanical cardiac performance for relatively long periods of time (24-48 hours or even more) so as to provide an improved Holter system.

It is thus provided a non-invasive apparatus adapted to monitor parameters indicative of heart performance, said apparatus comprising:
At least one sensor adapted to continuously sense factors correlated with blood flow and collect data related to the flow of blood, said sensor is adapted to be positioned adjacent to a peripheral blood vessel;
a processor adapted to receive the collected data from said at least one sensor and calculate the parameters indicating heart performance;
a monitor on which the parameters indicating heart performance are displayed.

Furthermore, in accordance with another preferred embodiment of the present invention, said at least one sensor, said processor and said monitor are incorporated in a portable device, said portable device is adapted to be mounted on a body part in which a peripheral blood vessel passes.

Furthermore, in accordance with another preferred embodiment of the present invention, said portable device is worn on a wrist.

Furthermore, in accordance with another preferred embodiment of the present invention, said peripheral blood vessel is selected from a group of blood vessels such as a radial artery, a cubital artery, a tibial artery, a femoral artery and a carotid artery.

Furthermore, in accordance with another preferred embodiment of the present invention, one of said at least one sensor is a pressure sensor.

Furthermore, in accordance with another preferred embodiment of the present invention, one of said at least one sensor is an electromagnetic sensor.

Furthermore, in accordance with another preferred embodiment of the present invention, said parameters indicating heart performance comprises stroke volume, cardiac output, and stroke work.

Furthermore, in accordance with another preferred embodiment of the present invention, said apparatus further comprises a means for transmitting the parameters indicating heart performance to external units.

Furthermore, in accordance with another preferred embodiment of the present invention, said means for transmitting is telemetry.

Furthermore, in accordance with another preferred embodiment of the present invention, said apparatus further comprises alarm means adapted to alert on irregularities in the blood flow.

Furthermore, in accordance with another preferred embodiment of the present invention, the alarm means is selected from the group consisting of audible alarm, visible alarm or vibrating alarm.

Furthermore, in accordance with another preferred embodiment of the present invention, the alarm is a beep sound.

Furthermore, in accordance with another preferred embodiment of the present invention, the alarm is a flashing light.

Furthermore, in accordance with another preferred embodiment of the present invention, said apparatus is provided with a socket through which the parameters indicating heart performance are electrically transmitted to an external processor, said external processor is electrically connected to a Holter system adapted to collect ECG signals.

Furthermore, in accordance with another preferred embodiment of the present invention, said external processor is provided with at least one algorithm, the algorithm is adapted correlate the data related to blood flow with the ECG signals in order to eliminate artifacts in the ECG signals.

Furthermore, in accordance with another preferred embodiment of the present invention, said processor is electrically communicating with a computer, said computer is provided with a storing means.

Furthermore, in accordance with another preferred embodiment of the present invention, one of said at least one sensor is temperature sensor.

Furthermore, in accordance with another preferred embodiment of the present invention, one of said at least one sensor is an ionic sensor said ionic sensor is adapted to continuously measure changes in blood resistance due to a current induced on said blood vessel by a source electrode, and is adapted to interpret blood flow velocity from the blood resistance measurement.

Furthermore, in accordance with another preferred embodiment of the present invention, one of at least one sensor is an inertial sensor that detects the mechanical motion of said apparatus.

It is thus also provided a non-invasive apparatus adapted to monitor parameters indicative of blood flow, said apparatus comprising:
At least one magnet adapted to be mounted on a limb in which blood vessels pass, and adapted to induce a magnetic flux substantially normal to the direction of the blood flow in said blood vessels;
at least two electrodes adjacent to said at least one magnet and adapted to be contiguously coupled to said limb, said at least two electrodes adapted to continuously sense induced voltage that correspond the flow of blood in said blood vessels;
a processor adapted to receive values of said induced voltage from said at least two electrodes and calculate parameters indicating blood flow;
a monitor on which the parameters indicating blood flow are displayed.

Furthermore, in accordance with another preferred embodiment of the present invention, said at least one magnet is incorporated in a resilient material shaped as a bracelet that substantially fits a person's wrist.

Furthermore, in accordance with another preferred embodiment of the present invention, a plurality of electrodes are embedded in said resilient material, and wherein said plurality of electrodes are provided with contact points that are adapted to sense voltage in a plurality of points on a line that circles about said limb.

Furthermore, in accordance with another preferred embodiment of the present invention, a band is provided adjacent to said resilient material, said band is adapted to establish and assure good contact between said plurality of contact points and said limb.

Furthermore, in accordance with another preferred embodiment of the present invention, said at least one magnet produces substantially pseudo-uniform magnetic filed across said limb.

Furthermore, in accordance with another preferred embodiment of the present invention, said at least one magnet produces substantially rotative pseudo-uniform magnetic filed across said limb.

Furthermore, in accordance with another preferred embodiment of the present invention, said processor is coupled to said at least one magnet.

Furthermore, in accordance with another preferred embodiment of the present invention, said induced voltage that is sensed by said at least two electrodes is amplified by differential preamplifiers.

Furthermore, in accordance with another preferred embodiment of the present invention, digitized results of the amplified induced voltage are transferred to said processor through a multiplexer and an A/D converter.

Furthermore, in accordance with another preferred embodiment of the present invention, said processor is provided with algorithms that comprises mathematical formulations through which hemodynamic indexes are resulted.

Furthermore, in accordance with another preferred embodiment of the present invention, said processor is provided with an algorithm based on a 2D intensity image reconstruction that outputs cross sectional images of said blood vessels, and wherein an intensity parameter in said image is indicative of blood flow velocity.

Furthermore, in accordance with another preferred embodiment of the present invention, said processor is provided with a keyboard through which instruction are transferred to said processor.

Furthermore, in accordance with another preferred embodiment of the present invention, said keyboard and said processor are miniature enough so as to be mounted with said at leas one magnet on said limb.

Furthermore, in accordance with another preferred embodiment of the present invention, said monitor is a liquid crystal display that is coupled to said processor and is mounted on said limb adjacent to said at least one magnet.

Furthermore, in accordance with another preferred embodiment of the present invention, said apparatus further comprises a means for transmitting the parameters indicating heart performance to external units.

Furthermore, in accordance with another preferred embodiment of the present invention, said means for transmitting is telemetry.

Furthermore, in accordance with another preferred embodiment of the present invention, one of said external units is a storing means.

Furthermore, in accordance with another preferred embodiment of the present invention, said apparatus further comprises alarm means adapted to alert on irregularities in the blood flow.

Furthermore, in accordance with another preferred embodiment of the present invention, the alarm means is selected from the group consisting of audible alarm, visible alarm or vibrating alarm.

Furthermore, in accordance with another preferred embodiment of the present invention, the alarm is a beep sound.

Furthermore, in accordance with another preferred embodiment of the present invention, the alarm is a flashing light.

Furthermore, in accordance with another preferred embodiment of the present invention, said apparatus is provided with means through which the parameters indicating blood flow are electrically transmitted to an external processor, said external processor is electrically connected to a Holter system adapted to collect ECG signals.

Furthermore, in accordance with another preferred embodiment of the present invention, said external processor is provided with at least one algorithm, the algorithm is adapted to correlate the data related to blood flow with the ECG signals in order to eliminate artifacts in the ECG signals.

Furthermore, in accordance with another preferred embodiment of the present invention, said apparatus is powered by an adjacent power supply.

Furthermore, in accordance with another preferred embodiment of the present invention, said apparatus is further provided with a temperature sensor adapted to be used as calibration means for changes in temperatures.

Furthermore, in accordance with another preferred embodiment of the present invention, said apparatus is further provided with an ionic sensor, said ionic sensor is adapted to continuously measure changes in blood resistance due to a current induced on said blood vessel by a source electrode, and is adapted to interpret blood flow velocity from the blood resistance measurement.

Furthermore, in accordance with another preferred embodiment of the present invention, said apparatus is further provided with an inertial sensor that detects the mechanical motion of said apparatus.

Furthermore, in accordance with another preferred embodiment of the present invention, said apparatus is further provided with means for temporary venous blood occlusion.
It is thus also provided A non-invasive apparatus adapted to monitor parameters indicative of blood flow velocity, said apparatus comprising:
At least one current source electrode adapted to be mounted on a limb in which blood vessels pass, and adapted to induce a current flux substantially normal to the direction of the blood flow in said blood vessels;
at least two electrodes positioned in a predetermined distance from said at least one current source and adapted to be contiguously coupled to said limb, one of said at least two electrodes is adapted to continuously sense blood resistance and another one of said at least two electrodes is adapted to act as a reference electrode so that said at least two electrodes are adapted to measure the resistance of blood in said blood vessels;
time measuring means adapted to determine time interval between a time in which a current pulse is induced by said at least one current source electrode and a time in which a difference in resistance is sensed by said at least two electrodes;
a processor adapted to receive resistance values from said at least two electrodes and the time intervals from said time measuring means, said processor is adapted to calculate blood flow velocity;

a monitor on which blood flow velocities are displayed.

It is thus also provided a non-invasive method for monitoring parameters indicative of heart performance, said method comprising:
providing a non-invasive apparatus that comprises
   at least one sensor adapted to continuously sense factors correlated with blood flow and collect data related to the flow of blood, said sensor is adapted to be positioned adjacent to a peripheral blood vessel;
   a processor adapted to receive the collected data from said at least one sensor and calculate the parameters indicating heart performance;
   a monitor on which the parameters indicating heart performance are displayed;
mounting said apparatus on a patient's limb.

Furthermore, in accordance with another preferred embodiment of the present invention, said method further comprises transferring said parameters indicating heart performance to a medical center.

Furthermore, in accordance with another preferred embodiment of the present invention, said method further comprises alerting when irregularities in the blood flow are detected.

Furthermore, in accordance with another preferred embodiment of the present invention, said method further comprises attaching a Holter device to the patient and synchronizing said parameters indicating heart performance with results from measurements performed by said Holter device.

It is thus also provided a non-invasive method for monitoring parameters indicative of blood flow, said method comprising:
providing a non-invasive apparatus that comprises
   at least one magnet adapted to be mounted on a limb in which blood vessels pass, and adapted to induce a magnetic flux substantially normal to the direction of the blood flow in said blood vessels;
   at least two electrodes adjacent to said at least one magnet and adapted to be contiguously coupled to said limb, said at least two electrodes adapted to continuously sense induced voltage that correspond the flow of blood in said blood vessels;
   a processor adapted to receive values of said induced voltage from said at least two electrodes and calculate parameters indicating blood flow;
   a monitor on which the parameters indicating blood flow are displayed;
mounting said non-invasive apparatus on the patient's limb.

Furthermore, in accordance with another preferred embodiment of the present invention, said method further comprises calculating parameters indicating heart performance from said parameters indicating blood flow.

Furthermore, in accordance with another preferred embodiment of the present invention, said method further comprises transferring said parameters indicating heart performance to a medical center.

Furthermore, in accordance with another preferred embodiment of the present invention, said method further comprises alerting when irregularities in the blood flow are detected.

Furthermore, in accordance with another preferred embodiment of the present invention, said method further comprises transferring said parameters indicating blood flow to an external computer unit.

Furthermore, in accordance with another preferred embodiment of the present invention, said method further comprises attaching a Holter device to the patient and synchronizing said parameters indicating blood flow with results of measurements performed in said Holter device.

Furthermore, in accordance with another preferred embodiment of the present invention, said method further comprise the following steps for zeroing measurements in said apparatus
occluding temporarily venous blood in order to establish a pure artery flow;
measuring the decay in artery flow;
modeling the rate of artery decay;

estimating an average artery flow before occlusion.

### BRIEF DESCRIPTION OF THE FIGURES

FIG. 1 illustrates a noninvasive device for monitoring heart mechanical performance in accordance with a preferred embodiment of the present. invention, worn on a wrist and synchronized with Holter system.

FIG. 2 illustrates a cross-sectional view of a monitoring device in accordance with another preferred embodiment of the present invention provided with an electromagnetic sensor.

FIG. 3 illustrates a schematic diagram of an optional configuration of an electrical circuitry to which an electromagnetic sensor in accordance with a preferred embodiment of the present invention, is electrically connected.

FIG. 4 illustrates a lateral cross section of an ionic flow meter incorporated in a monitoring device in accordance with yet another preferred embodiment of the present invention.

FIG. 5 illustrating a graph showing the resistance measurement after a pulse is given in accordance with a measurement made using the monitoring device shown in FIG. 4.

### DETAILED DESCRIPTION OF THE INVENTION AND FIGURES

The present invention provides a noninvasive device and method for monitoring the mechanical performance of the heart muscle in a continuous manner. The noninvasive monitoring device is relatively small in dimensions, therefore portable and may be designed as a bracelet that may be worn on the wrist of a patient and continuously monitor information and store the information. As indicated herein before, the inventors of the present invention showed a clear and distinct correlation between indexes of heart performance measured centrally and peripherally, therefore, the convenience of a bracelet-like device is apparent.

There is an acceptance in cardiology that reduction in the global left ventricular function in ischemic heart diseases is due to lesions proximal to the coronary arteries and very severe ischemia. Therefore, ST segment depression, which is a hallmark of ischemia associated with the mechanical estimate global left ventricular function may provide a unique method for detecting ischemia in general and severe ischemia with poor prognosis in particular, thus provide a noninvasive stratification of patients. The stroke work (SW) is the external work performed by the left ventricle and is calculated as the area of the pressure/volume loop. It can be approximated as the product of SV and mean arterial pressure; thus the stroke work integrates the two determinants of perfusion: flow and pressure. Since the measurements of the monitoring device of the present invention are preferably performed on a peripheral artery, the peripheral SV is to be estimated. The peripheral SV (PSV) estimation is provided from velocity signals multiplied by the diameter of the artery. It is assumed that the diameter of the radial artery or any other peripheral artery that is used for the present invention does not change significantly from beat to beat and the PSV is calculated from multiplying the integrated velocity curve by the flow and the radial artery area. The peripheral CO is calculated from multiplying the PSV by the heart rate and finally, the peripheral stroke work is calculated from multiplying PSV by the mean arterial pressure.

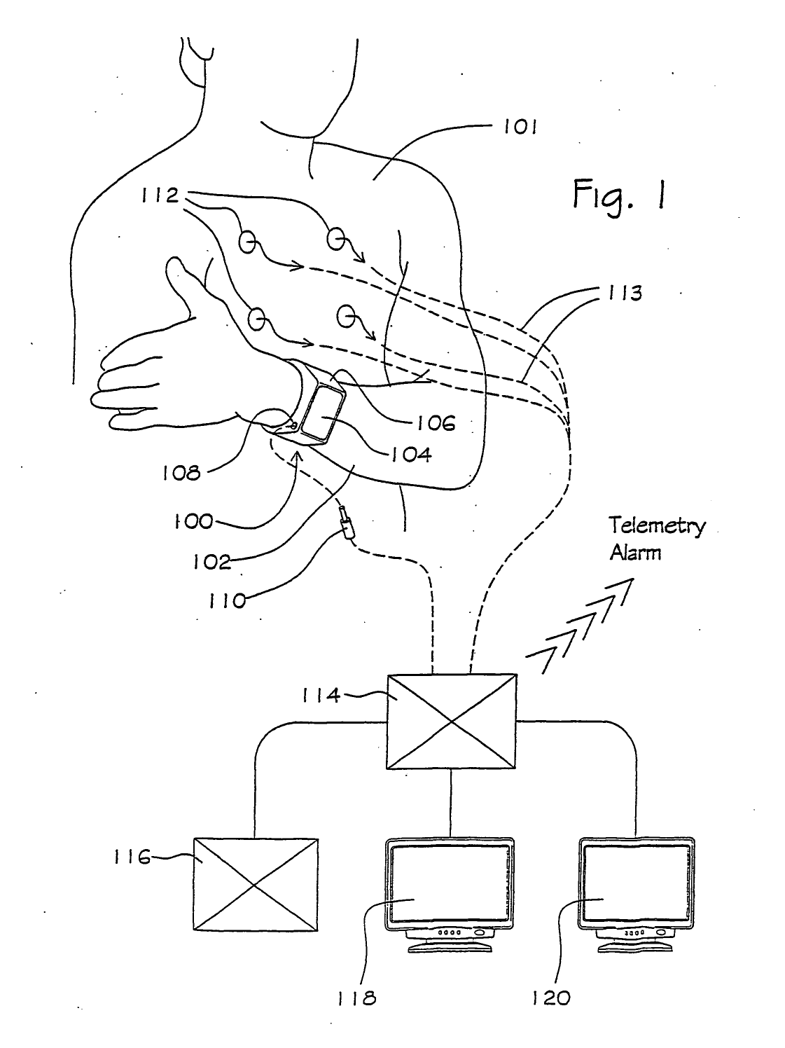

Reference is now made to FIG. 1 illustrating a noninvasive device for monitoring heart mechanical performance in accordance with a preferred embodiment of the present invention, worn on a wrist and synchronized with a Holter. A monitoring device 100 in the shape of a bracelet is preferably worn on a wrist 102 of a patient 101. Monitoring device 100 comprises a first portion 104 in which mainly electronic components are installed. A second portion 106 is designated for mounting the device on the wrist and securing it on wrist 102 and it comprises sensors or any additional component needed for beat-to-beat monitoring as will be comprehensively explained herein after. In a preferred embodiment shown in FIG. 1, two sensors are provided: a detector such as a Doppler sensor or an electromagnetic sensor (the electromagnetic sensor will be discussed in details later), which detects the speed of blood through the brachial or radial artery, and a pressure sensor. It should be mentioned that incorporating the two sensors together is performed from convenience considerations; however, both sensors may be placed on different limbs or organs. From both sensors, the pressure and the flow of blood may be estimated and indexes of cardiac function may be provided using the calculations indicated herein above. The use of more than one sensor is designated to enhance the overall information over the cardiac performance. First portion 104 is provided with a socket 108 to which a processor 114 may be connected using a corresponding connector 110 so that information stored by the electronic components of monitoring device 100 may be transferred or printed for inspection and further analysis and evaluation. Monitoring device 100 is portable and may be carried on the wrist of the patient so that the measurements are continuously collected while the patient may attend to other matters.

Beat-to-beat ambulatory monitoring of the heart performance that is achieved using sensors that are positioned adjacent to a peripheral artery such as the radial or the cubital artery is an essence of the noninvasive monitoring. Using mathematical manipulation on information gathered from the sensors, parameters such as peripheral blood flow, stroke volume, cardiac output and stroke power are calculated (as described herein before), providing indication on the mechanical performance of the heart. Synchronization of the mechanical information of hemodynamic indexes with ECG signals that may be provided, for example, using a Holter system, an overall accurate evaluation regarding the heart condition may be established. Serious drawbacks of Holter systems are artifacts that are not reflecting real heart performance. The combination of the sensors of the present invention with preferably a Holter system comprises an improved Holter system that eliminates these artifacts. The resulting improved Holter of the present invention has all the advantages to overcome the drawbacks of the existing Holter systems. In order to synchronize the results from the pressure sensor and the Doppler sensor with ECG results that may be established by a Holter, electrodes 112 are placed on patient's 101 chest in a standard manner. Electrodes 112, which are standard electrodes used for ECG measurements, are electrically connected by wires 113 to processor 114 in which the results may be transferred in a standard manner to a Holter device 116. The communication between the sensors in monitoring device 100 and Holter device 116 may be cordless. The results formulated from the pressure and Doppler sensors and the ECG may be displayed on a display 118 or transferred to a computer 120 for further analysis, storage or evaluation. Among the evaluation of the condition of the patient, the electronic components of the device may be provided with specific parameters that facilitate evaluation of drug management. Moreover, ischemic signs of the patient's heart may be registered and the patient may be alerted accordingly.

Other alternative embodiments, in which other sensors that monitor heart activity are used, may be synchronized with the sensors of the present invention in order to produce new and advanced systems for monitoring electrical activity of the heart. The alternative embodiments are improved systems for monitoring heart activity since they provide additional information that is established through blood velocity data. Artifacts that are prone in the available systems for monitoring heart activity are eliminated. The alternative embodiments are covered by the scope of the present invention.

The invention further provides a method and apparatus for calculating the systemic stroke volume and the cardiac output from the peripheral SV index value. In the present method, the CO is calibrated and determined by known calculation methods from the mechanical ECG-Holter collected from Holter device 116. The ECG-Holter mechanical information synchronized with the ischemic event detected by the sensors contemporaneous indicate any change in the patient's cardiac performance. The electronic components of the noninvasive device for monitoring heart mechanical performance of the present invention may also use algorithms that may be provided with models for heart performance as well as parameters on the previous condition of the patient. The models and other parameters may facilitate in the establishment of real-time continuous medical assessment and evaluation of multi parameters and varying clinical conditions simultaneously. In addition, drug management may be also continuously evaluated and alert the patient by audio or visual means the need to seek medical help.

It is known in the art that the presence of magnetic field in the vicinity of a blood vessel, cause electrical currents that are induced by the ions flowing in the blood. The induced electrical current is proportional to the flow of blood in the vessel. Since the only source of flow in the wrist area are arteries and veins, it is suggested in the present invention to use an electromagnetic sensor as an additional embodiment of the present invention. This will reduce the interference sources. The noninvasive electromagnetic blood flow meter of the present invention is based on applying a magnetic field externally, and measuring voltage drops by means of external electrodes. The voltage drop measured by the electrodes is proportional to the blood velocity the calculation of the blood velocity is based on well-known mathematical formulations as well as on a cross section model (2D image reconstruction) as will be discussed herein after.

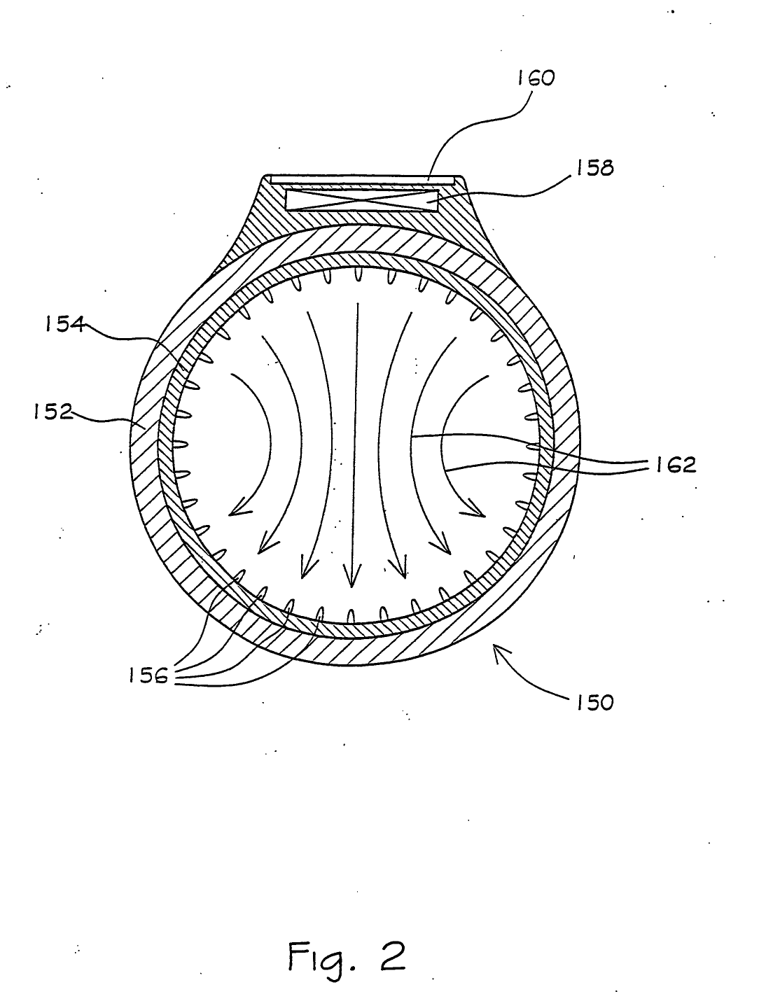

Reference is now made to FIG. 2, illustrating a cross-sectional view of a monitoring device in accordance with another preferred embodiment of the present invention provided with an electromagnetic sensor. A monitoring device 150 in the shape of a bracelet that fits an average person's wrist is made of a flexible plastic material incorporated with a flexible magnet 152. It is important to notice that monitoring device 150 may be mounted on the upper arm of the patient, on the ankle or on the neck. In all those body parts, peripheral blood vessels pass through and their blood velocity may be measured with minimal interruptions. Flexible magnet 152 produces a pseudo-uniform (non-gradient) magnetic field (indicated by arrows 162). The magnetic field is normal to the blood flow direction when monitoring device 150 is mounted as a bracelet on a user's wrist (similarly to the embodiment shown in FIG. 1). In an alternative embodiment of the present invention, the magnetic field that produced by flexible magnet 150 may be also a rotative pseudo-uniform magnetic field so that the magnetic field is in a transversal direction in respect to the blood flow direction.

The circumference of flexible magnet 152 is provided with a band 154 of flexible material that holds the magnet in place. The inner circumference of band 154 is provided with a plurality of electrodes 156 that are directed inwardly and held in contact with the body part on which monitoring device 150 is mounted. Electrodes 156 are multipoint surface sense electrodes that sense the dropout voltage produced by the magnetic and the blood flow-induced electric current. The multipoint surface electrodes increase the signal-to-noise ratio of the device. The electrodes are combined using a signal processing algorithm in order to obtain a differential measurement enhancing the signal to noise ratio. A database of signals is used in order to decode the flow information based on the periodicity of the signals and employing a database of feasible signals. The decoded signal is filtered over several periods and then analyzed for detecting possible changes. The signal acquired by electrodes 156 indicates the estimation of the artery and vein blood flow. The measurements acquired by the electrodes are transferred electrically to a microprocessor 158 that processes the results from the electrodes. The microprocessor is provided with algorithms that process the signal, condition the measurement, analyze the measurement and may compare the measurement with models of cardiac mechanical performance and to other measurements that were taken previously from the patient. As mentioned herein before, the results may be synchronized with ECG results of the patient that may be fed into microprocessor 158. Systemic stroke volume and cardiac output may be calculated from the peripheral SV index value. In the present method, the CO is calibrated and determined by known calculation methods from the mechanical ECG-Holter collected from an independent Holter device.

It is noted that all parts of the bracelet that are in contact with body parts are made of materials that are compatible with the human skin and may not cause any irritant reaction. The microprocessor is provided with a storing means so that the results from the continuous measurement may be stored for surveillance on the condition of the patient for a long period of time.

In an alternative embodiment, the differential estimation of the artery and blood flow is based on 2D intensity image reconstruction algorithm in a similar manner as done in CT imaging. The output reconstruction is 2D intensity image of the arteries and veins cross sections while the intensity is dependent on the blood flow velocity. The vessel's squared cross-section multiplied by the flow velocity will provide the volumetric flow. Processed results may be displayed on a liquid crystal display, LCD 160.

As mentioned herein before, the blood vessels in the wrist area are veins and arteries. It follows that the meter can measure instantaneously variation of the flow but not artery flow by itself. In order to estimate the flow of blood in the artery, it is required to temporarily stop the blood flow in the vein by applying external pressure or by any other method. During the period of time in which the vein flow is occluded, the decay of the artery flow is measured. This measurement may be used for zeroing the sensor and may be used in a model for estimating the steady artery flow using preferably the following steps:
1. occlude a peripheral vein in order to establish a pure artery flow;
2. measure the decay in artery flow due to occlusion of veins;
3. model the rate of artery decay;
4. estimate an average artery flow before occlusion.

Monitoring device 150 may be a standalone apparatus for monitoring blood flow alone and may be incorporated with a Holter system in order to established an improved Holter apparatus.

In additional embodiments of the present invention, the apparatus is provided with additional miniature sensors in order to eliminate influence of factors in the surroundings. One of the factors that may influence the device is the temperature. A temperature sensor may be incorporated in monitoring device 150 so that the temperature is known at any minute. Microprocessor 158 is then provided with an algorithm that correlated the temperature measurement to the blood flow measurement so that the device is calibrated at any given time.

In yet another embodiment of the present invention, monitoring device 150 is provided with at least one miniature inertia sensor. Inertia sensors allow correlating the reading of the device with the movements of the body part on with monitoring device 150 is mounted. In this way, the artifacts that are not related to the flow measurement will be eliminated. From the sampled signal, only the AC component is suitable for the flow waveform estimation since the blood flow of the vein and the artery are approximately equal after a long period of time.

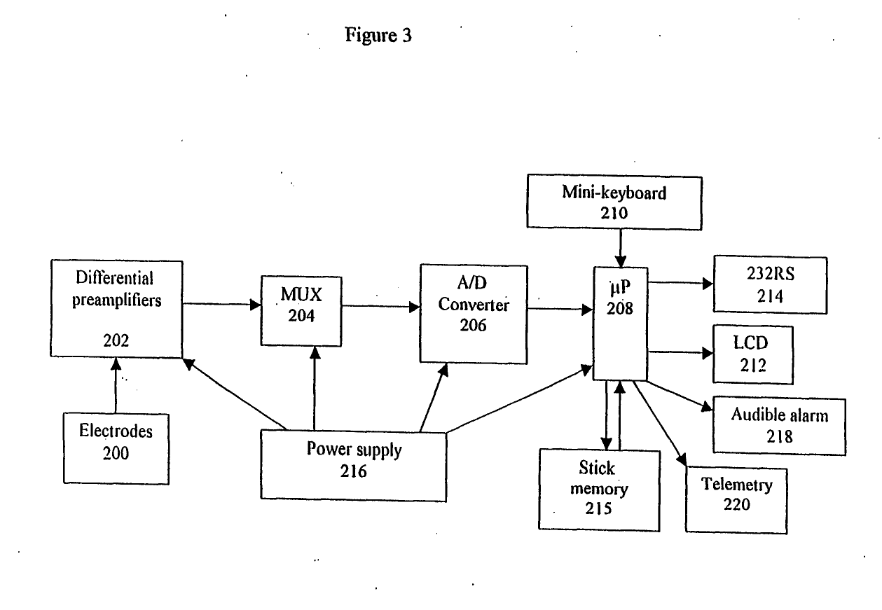

Reference is now made to FIG. 3 illustrating a schematic diagram of an optional configuration of an electrical circuitry to which an electromagnetic sensor in accordance with a preferred embodiment of the present invention is electrically connected. Surface multipoint electrodes 200 sense and collect information in the shape of signals. The information signals are transferred to differential preamplifiers 202 that amplify the received signals. The signals are than transferred to multiplexer 204 and digitized in an A/D converter 206. The data is now being processed by a microprocessor 208 that is provided with preferably mini-keyboard 210 through which the user may communicate with the device. Microprocessor 208 is provided with algorithms that comprise a set of steps including mathematical formulations indicated herein above, in order to provide the necessary hemodynamic indexes. Results or selected results may be displayed on a LCD 212, transferred to off-line processing or storage preferably through RS232 214, or stored on stick memory 215. RS232 may transmit the information to a medical facility where the information may be combined with other information sources such as Holter or an independent measurement of blood flow. In a preferred embodiment, the system is provided with alarm 218 and/or telemetry means 220. Alarm 218 may be an audible alarm, a visible alarm or vibration of the apparatus. In cases the signal-processing algorithm detects a possible irregular flow of blood, a beep may be heard through alarm 218, a flashing light may be lit or the apparatus may be vibrating in order to alert the patient or the staff in a medical facility of the finding. The measured signals or results of an analysis made by microprocessor 208 may be sent by telemetry means 220 to a Holter in case a combination of results is required or to any third apparatus for further use. A power unit 216 powers the components of the monitoring device.

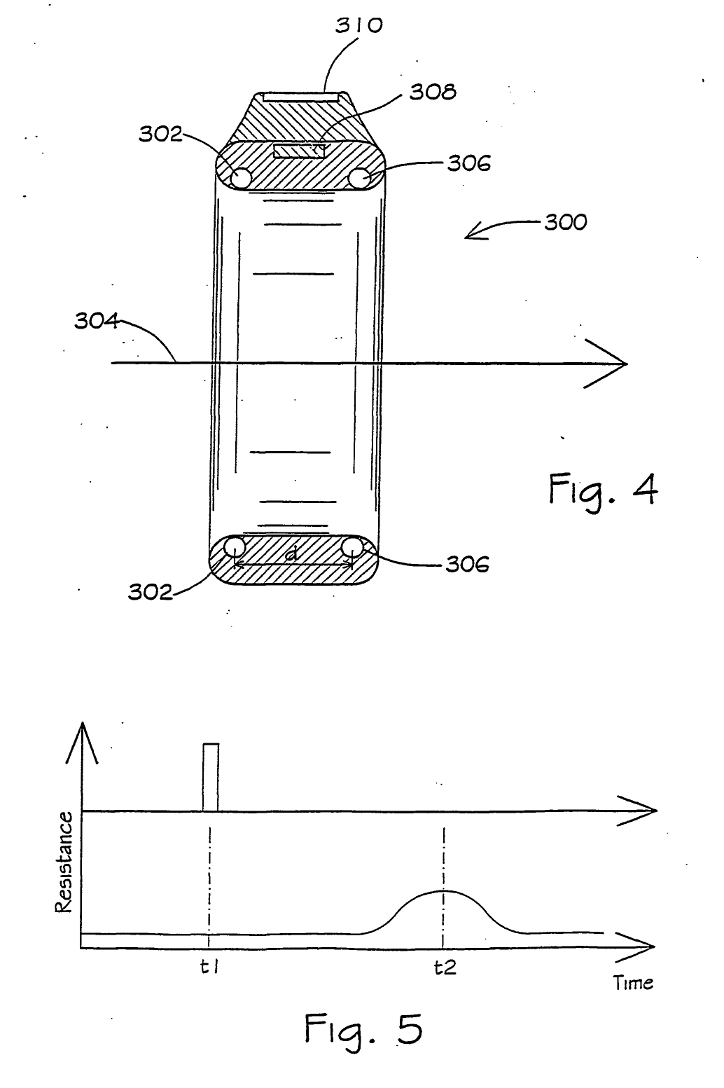

In another preferred embodiment of the present invention, an ionic flow sensor is incorporated in the monitoring device. Reference is now made to FIG. 4 illustrating a lateral cross section of an ionic flow meter incorporated in a monitoring device in accordance with yet another preferred embodiment of the present invention. Monitoring device 300 is shaped preferably as a bracelet similarly to monitoring device 100. Monitoring device 300 comprises a pulse current source 302 that is preferably a driving electrode that produces a pulsatile current. When monitoring device is mounted on preferably a wrist of a subject, the pulsatile current causes a separation of positive and negative charges that flows in the blood of the arteries and veins passing in the wrist area. By the well-known electrophoresis principle, the resistance of the volume surrounded by the source first decreases and then increased. The difference in resistance in the blood acts as a mark that moves according to the flow of blood so that marks are flowing in opposite directions by arteries and veins. An arrow 304 marks the direction of blood flow in the arteries. Alternatively, a magnet may be used as a source that produces a magnetic field across the arteries so that pulsatile magnetic filed produces a similar mark. Preferably two electrodes 306 that sense the resistance of the area encircled by the electrodes are incorporated in monitoring device 300. Two electrodes 306 are positioned in a predetermined distance d, in the direction of the artery blood flow 304, from pulse current source 302. The arrangement of pulse current source 302 and electrodes 306 in a predetermined distance is aimed to produce the mark by the source electrode and then detect it by the sense electrodes that are synchronized with the current or the magnetic pulse that flows in the blood stream. The time interval between the time the pulse emits from pulse current source 302 and produces the mark, and the time the mark reaches sense electrodes 306 where it is detected, is inversely proportional to the blood velocity.

In order to better understand the blood velocity estimation principle, reference is now made to FIG. 5 illustrating a graph showing the resistance measurement after a pulse is given in accordance with a measurement made using the monitoring device shown in FIG. 4. The vertical axis stands for the resistance in arbitrary units and the x-axis stands for the time. The upper part of the graph illustrates a typical pulse given by a pulse electrode and the lower part of the graph illustrates a typical measurement that is detected by the sense electrode. The indications, t₁ and t₂, indicate the time of current emission and the time of current detection, respectively. The calculation of blood velocity is given by V=d (t₂−t₁) where d is the physical distance between the source electrode and the sense electrode. The calculation is based on the assumption that the difference in resistance of blood in the vein can be neglected.

Returning to FIG. 4, monitoring device 300 further comprises electronic elements 308 in which the calculation is performed based on the results from sense electrodes 306 that transfers the resistance measurements similarly to monitoring device 100 and 200. Electronic elements 308 may preferably be electronically connected to an LCD screen 310 that display the results. In this embodiment, as in the previous ones, the results may be transferred to other units for further inspection and storage purposes.

It should be clear that the description of the embodiments and attached Figures set forth in this specification serves only for a better understanding of the invention, without limiting its scope as covered by the following claims.

It should also be clear that a person skilled in the art, after reading the present specification could make adjustments or amendments to the attached Figures and above described embodiments that would still be covered by the following claims.

1. A non-invasive apparatus adapted to monitor parameters indicative of heart performance, said apparatus comprising:
At least one sensor adapted to continuously sense factors correlated with blood flow and collect data related to the flow of blood, said sensor is adapted to be positioned adjacent to a peripheral blood vessel;
a processor adapted to receive the collected data from said at least one sensor and calculate the parameters indicating heart performance;
a monitor on which the parameters indicating heart performance are displayed.

2. The apparatus as claimed in claim 1, wherein said at least one sensor, said processor and said monitor are incorporated in a portable device, said portable device is adapted to be mounted on a body part in which a peripheral blood vessel passes.

3. The apparatus as claimed in claim 2, wherein said portable device is worn on a wrist.

4. The apparatus as claimed in claim 1, wherein said peripheral blood vessel is selected from a group of blood vessels such as a radial artery, a cubital artery, a tibial artery, a femoral artery and a carotid artery.

5. The apparatus as claimed in claim 1, wherein one of said at least one sensor is a pressure sensor.

6. The apparatus as claimed in claim 1, wherein one of said at least one sensor is an electromagnetic sensor.

7. The apparatus as claimed in claim 1, wherein said parameters indicating heart performance comprises stroke volume, cardiac output, and stroke work.

8. The apparatus as claimed in claim 1, wherein said apparatus further comprises a means for transmitting the parameters indicating heart performance to external units.

9. The apparatus as claimed in claim 8, wherein said means for transmitting is telemetry.

10. The apparatus as claimed in claim 1, wherein said apparatus further comprises alarm means adapted to alert on irregularities in the blood flow.

11. The apparatus as claimed in claim 10, wherein the alarm means is selected from the group consisting of audible alarm, visible alarm or vibrating alarm.

12. The apparatus as claimed in claim 10, wherein the alarm is a beep sound.

13. The apparatus as claimed in claim 10, wherein the alarm is a flashing light.

14. The apparatus as claimed in claim 1, wherein said apparatus is provided with a socket through which the parameters indicating heart performance are electrically transmitted to an external processor, said external processor is electrically connected to a Holter system adapted to collect ECG signals.

15. The apparatus as claimed in claim 14, wherein said external processor is provided with at least one algorithm, the algorithm is adapted correlate the data related to blood flow with the ECG signals in order to eliminate artifacts in the ECG signals.

16. The apparatus as claimed in claim 14, wherein said processor is electrically communicating with a computer, said computer is provided with a storing means.

17. The apparatus as claimed in claim 1, wherein one of said at least one sensor is temperature sensor.

18. The apparatus as claimed in claim 1, wherein one of said at least one sensor is an ionic sensor, said ionic sensor is adapted to continuously measure changes in blood resistance due to a current induced on said blood vessel by a source electrode, and is adapted to interpret blood flow velocity from the blood resistance measurement.

19. The apparatus as claimed in claim 1, wherein one of at least one sensor is an inertial sensor that detects the mechanical motion of said apparatus.

20. A non-invasive apparatus adapted to monitor parameters indicative of blood flow, said apparatus comprising:
At least one magnet adapted to be mounted on a limb in which blood vessels pass, and adapted to induce a magnetic flux substantially normal to the direction of the blood flow in said blood vessels;
at least two electrodes adjacent to said at least one magnet and adapted to be contiguously coupled to said limb, said at least two electrodes adapted to continuously sense induced voltage that correspond the flow of blood in said blood vessels;
a processor adapted to receive values of said induced voltage from said at least two electrodes and calculate parameters indicating blood flow;
a monitor on which the parameters indicating blood flow are displayed.

21. The apparatus as claimed in claim 20, wherein said at least one magnet is incorporated in a resilient material shaped as a bracelet that substantially fits a person's wrist.

22. The apparatus as claimed in claim 21, wherein a plurality of electrodes are embedded in said resilient material, and wherein said plurality of electrodes are provided with contact points that are adapted to sense voltage in a plurality of points on a line that circles about said limb.

23. The apparatus as claimed in claim 22, wherein a band is provided adjacent to said resilient material, said band is adapted to establish and assure good contact between said plurality of contact points and said limb.

24. The apparatus as claimed in claim 20, wherein said at least one magnet produces substantially pseudo-uniform magnetic filed across said limb.

25. An apparatus as claimed in claim 20, wherein said at least one magnet produces substantially rotative pseudo-uniform magnetic filed across said limb.

26. The apparatus as claimed in claim 20, wherein said processor is coupled to said at least one magnet.

27. The apparatus as claimed in claim 20, wherein said induced voltage that is sensed by said at least two electrodes is amplified by differential preamplifiers.

28. The apparatus as claimed in claim 27, wherein digitized results of the amplified induced voltage are transferred to said processor through a multiplexer and an A/D converter.

29. The apparatus as claimed in claim 20, wherein said processor is provided with algorithms that comprises mathematical formulations through which hemodynamic indexes are resulted.

30. The apparatus as claimed in claim 20, wherein said processor is provided with an algorithm based on a 2D intensity image reconstruction that outputs cross sectional images of said blood vessels, and wherein an intensity parameter in said image is indicative of blood flow velocity.

31. The apparatus as claimed in claim 20, wherein said processor is provided with a keyboard through which instruction are transferred to said processor.

32. The apparatus as claimed in claim 31, wherein said keyboard and said processor are miniature enough so as to be mounted with said at leas one magnet on said limb.

33. The apparatus as claimed in claim 20, wherein said monitor is a liquid crystal display that is coupled to said processor and is mounted on said limb adjacent to said at least one magnet.

34. The apparatus as claimed in claim 20, wherein said apparatus further comprises a means for transmitting the parameters indicating heart performance to external units.

35. The apparatus as claimed in claim 34, wherein said means for transmitting is telemetry.

36. The apparatus as claimed in claim 34, wherein one of said external units is a storing means.

37. The apparatus as claimed in claim 20, wherein said apparatus further comprises alarm means adapted to alert on irregularities in the blood flow.

38. The apparatus as claimed in claim 37, wherein the alarm means is selected from the group consisting of audible alarm, visible alarm or vibrating alarm.

39. The apparatus as claimed in claim 37, wherein the alarm is a beep sound.

40. The apparatus as claimed in claim 37, wherein the alarm is a flashing light.

41. The apparatus as claimed in claim 20, wherein said apparatus is provided with means through which the parameters indicating blood flow are electrically transmitted to an external processor, said external processor is electrically connected to a Holter system adapted to collect ECG signals.

42. The apparatus as claimed in claim 41, wherein said external processor is provided with at least one algorithm, the algorithm is adapted to correlate the data related to blood flow with the ECG signals in order to eliminate artifacts in the ECG signals.

43. The apparatus as claimed in claim 20, wherein said apparatus is powered by an adjacent power supply.

44. The apparatus as claimed in claim 20, wherein said apparatus is further provided with a temperature sensor adapted to be used as calibration means for changes in temperatures.

45. The apparatus as claimed in claim 20, wherein said apparatus is further provided with an ionic sensor, said ionic sensor is adapted to continuously measure changes in blood resistance due to a current induced on said blood vessel by a source electrode, and is adapted to interpret blood flow velocity from the blood resistance measurement

46. The apparatus as claimed in claim 20, wherein said apparatus is further provided with an inertial sensor that detects the mechanical motion of said apparatus.

47. The apparatus as claimed in claim 20, wherein said apparatus is further provided with means for temporary venous blood occlusion.

48. A non-invasive apparatus adapted to monitor parameters indicative of blood flow velocity, said apparatus comprising:
At least one current source electrode adapted to be mounted on a limb in which blood vessels pass, and adapted to induce a current flux substantially normal to the direction of the blood flow in said blood vessels;
at least two electrodes positioned in a predetermined distance from said at least one current source and adapted to be contiguously coupled to said limb, one of said at least two electrodes is adapted to continuously sense blood resistance and another one of said at least two electrodes is adapted to act as a reference electrode so that said at least two electrodes are adapted to measure the resistance of blood in said blood vessels;
time measuring means adapted to determine time interval between a time in which a current pulse is induced by said at least one current source electrode and a time in which a difference in resistance is sensed by said at least two electrodes;
a processor adapted to receive resistance values from said at least two electrodes and the time intervals from said time measuring means, said processor is adapted to calculate blood flow velocity;
a monitor on which blood flow velocities are displayed.

49. A non-invasive method for monitoring parameters indicative of heart performance, said method comprising:
providing a non-invasive apparatus that comprises
at least one sensor adapted to continuously sense factors correlated with blood flow and collect data related to the flow of blood, said sensor is adapted to be positioned adjacent to a peripheral blood vessel;
a processor adapted to receive the collected data from said at least one sensor and calculate the parameters indicating heart performance;
a monitor on which the parameters indicating heart performance are displayed;
mounting said apparatus on a patient's limb.

50. The method as claimed in claim 49, wherein said method further comprises transferring said parameters indicating heart performance to a medical center.

51. The method as claimed in claim 49, wherein said method further comprises alerting when irregularities in the blood flow are detected.

52. The method as claimed in claim 49, wherein said method further comprises attaching a Holter device to the patient and synchronizing said parameters indicating heart performance with results from measurements performed by said Holter device.

53. A non-invasive method for monitoring parameters indicative of blood flow, said method comprising:
providing a non-invasive apparatus that comprises
at least one magnet adapted to be mounted on a limb in which blood vessels pass, and adapted to induce a magnetic flux substantially normal to the direction of the blood flow in said blood vessels;
at least two electrodes adjacent to said at least one magnet and adapted to be contiguously coupled to said limb, said at least two electrodes adapted to continuously sense induced voltage that correspond the flow of blood in said blood vessels:
a processor adapted to receive values of said induced voltage from said at least two electrodes and calculate parameters indicating blood flow;
a monitor on which the parameters indicating blood flow are displayed;
mounting said non-invasive apparatus on the patient's limb.

54. The method as claimed in claim 53, wherein said method further comprises calculating parameters indicating heart performance from said parameters indicating blood flow.

55. The method as claimed in claim 53, wherein said method further comprises transferring said parameters indicating heart performance to a medical center.

56. The method as claimed in claim 53, wherein said method further comprises alerting when irregularities in the blood flow are detected.

57. The method as claimed in claim 53, wherein said method further comprises transferring said parameters indicating blood flow to an external computer unit.

58. The method as claimed in claim 57, wherein said method further comprises attaching a Holter device to the patient and synchronizing said parameters indicating blood flow with results of measurements performed in said Holter device.

59. The method as claimed in claim 53, wherein said method further comprises the following steps for zeroing measurements in said apparatus
occluding temporarily venous blood in order to establish a pure artery flow;
measuring the decay in artery flow;
modeling the rate of artery decay;
estimating an average artery flow before occlusion.

60. A non-invasive apparatus adapted to monitor parameters indicative of heart performance substantially as described in the above specifications, attached Figures and appending claims.

61. A non-invasive apparatus adapted to monitor parameters indicative of blood flow substantially as described in the above specifications, attached Figures and appending claims.

62. A non-invasive method for monitoring parameters indicative of heart performance substantially as described in the above specifications, attached Figures and appending claims.

63. A non-invasive method for monitoring parameters indicative of blood flow substantially as described in the above specifications, attached Figures and appending claims.

64. A non-invasive apparatus adapted to monitor parameters indicative of blood flow velocity substantially as described in the above specifications, attached Figures and appending claims.
